(19) United States
(12) Patent Application Publication
(10) Pub. No.: US 2023/0241255 A1
(43) Pub. Date: Aug. 3, 2023

(54) MAGNETIC RESONANCE CONTRAST AGENTS AND METHODS THEREOF

(71) Applicant: THE TEXAS A&M UNIVERSITY SYSTEM, College Station, TX (US)

(72) Inventors: Wei XU, College Station, TX (US); Leisha MARTIN, College Station, TX (US)

(21) Appl. No.: 17/588,458

(22) Filed: Jan. 31, 2022

(51) Int. Cl. A61K 49/18; A61B 5/055

(52) U.S. Cl. CPC A61K 49/183; A61B 5/055; A61K 49/1857; A61K 49/1875

(57) ABSTRACT

The present disclosure provides contrast agent compositions comprises a plurality of nanoparticles, such as nanoparticles comprises an iron nanoparticle such as an iron nitride. The disclosure also provides methods for magnetic resonance imaging of the contrast agent compositions as well as other methods of performing magnetic resonance imaging. Further, the disclosure provides kits comprising a contrast agent composition.

### BACKGROUND AND SUMMARY OF THE INVENTION

Techniques involving magnetic resonance imaging (MRI) are widely used in biomedical and clinical research. MRI scanners are the instruments employed in these techniques and are utilized to capture images of organs and tissues of the body using magnetic fields and radio waves. However, due to the different nature of tissues in humans, MRI scanners often fail to capture clear images in many tissues without the assistance of exogenous contrast agents, which are orally or intravenously administrated to subjects prior to the scanning.

Importantly, MRIs are limited by the sensitivity of the contrast agents utilized for the scanning. Among FDA-approved contrast agents, Gadolinium(III)-based contrast agents are used in 40% of MRIs and 60% of neuro MRIs, which corresponds to approximately 40 million administrations worldwide. Almost 50 tons of gadolinium are administered annually to patients and the market for gadolinium-based contrast agents exceeds $1 billion U.S. dollars per year.

However, gadolinium-based contrast agents are problematic for multiple reasons. Gadolinium is a rare-earth element, and its availability is in decline due to mining and export restrictions. Furthermore, the incidence of reported adverse effects associated with gadolinium is increasing. In 2006, gadolinium-based contrast agents were determined to be undesirably associated with nephrogenic system fibrosis in patients. In addition, it has been shown that gadolinium persists in the body for extended periods of time and gadolinium has been found to accumulate in the brain.

High-relaxivity materials are desirable and can be obtained from higher iron concentrations and better crystalline structures (e.g., demonstrating low or no defect states). Superparamagnetic particles' relaxivity can be described according to the quantum mechanical outer sphere theory, which posits that T2 relaxivity is proportional to the Mₛ (magnetic saturation value) and the superparamagnetic particle radius. The relaxivity (r₂) in the motional average regime, assuming a spherical particle, is given by:

r₂=(256π²γ²/405)κM_{S}²r²/D(1+L/r)=1/T2 (Eq. 1)

where γ is the proton gyromagnetic ratio; Mₛ and r are the saturation magnetization and effective radius of the magnetic nanoparticles, respectively; D is the diffusivity of water molecules; L is the thickness of an impermeable surface coating; and κ is the conversion factor (κ=V*=C_{Fe}, V* is the volume fraction, C_{Fe} is the concentration of Fe). These contrast agents demonstrate strong T1 relaxation properties and a strongly varying local magnetic field, which enhances T2 relaxation. Outer sphere theory modeling makes it clear that the ratio of r2/r1 increases with particle size, making smaller particles behave as superior T1-shortening agents, which is why SPIONs were initially developed as T2 agents and are visible as dark regions on MRI. However, ultrasmall SPIONs have superior T1-enhancing properties.

Therefore, there exists a need for new compositions and methods that can be used as contrast agents for imaging such as MRI. It is desirable for new contrast agents to have favorable magnetic properties and also an acceptable biosafety profile for human and veterinary use. Furthermore, it is desirable for new contrast agents to have MRI contrast properties that rival those of potentially toxic gadolinium-based options.

Accordingly, the present disclosure provides contrast agent compositions comprising a plurality of nanoparticles that can be utilized for MRIs. Furthermore, the present disclosure provides methods of performing magnetic resonance imaging in subjects, as well as kits comprising the contrast agent compositions.

The compositions and methods of the present disclosure provide several benefits compared to the current state of the art. First, the contrast agent compositions comprising iron have demonstrated T1 MRI contrast properties that are comparable to gadolinium-based contrast agents. Further, the described contrast agent compositions comprising iron can comprise a magnetic crystal that is 40 times more magnetic than iron oxide and 20% more magnetic than the strongest known magnetic alloy, iron-cobalt. In particular, the described contrast agent compositions have 45% more iron in their cores than magnetite superparamagnetic iron-based nanoparticles and may serve as “T1” or “T2” contrast agents, as either soft or hard magnetic material with negligible or high coercivity, depending on their size.

The superparamagnetic character of the described contrast agent compositions requires less magnetic field force generated by MRI scanner. Therefore, application of the described contrast agent compositions in MRI procedures can reduce the cost of MRI instruments, which makes MRI procedures possible for smaller hospitals and for developing countries.

Moreover, the contrast agent compositions can comprise different grain sizes for utilization as either a soft or a hard magnetic material having negligible or high coercivity which can be tailored to the specific application. Finally, the contrast agent compositions are environmentally friendly upon disposal in comparison to cobalt and rare earth materials, which can be associated with toxicity.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows a high resolution (HR) TEM image showing fringing and excellent crystallinity (scale bar is 5 nm).

FIG. 2 shows the proposed experimental design for evaluation in liver cancer.

FIG. 3 shows the proposed experimental design for evaluation in breast cancer.

FIG. 4 shows the proposed experimental design for evaluation in brain cancer. Locations of the guide screw were indicated by the dot above the eyes of the rat.

FIGS. 5A and 5B show evaluation of toxicity of the described contrast agents compared to control contrast agents. FIG. 5A shows assessment in cell cultures. FIG. 5B shows assessment in rats with chronic kidney disease (CKD).

### DETAILED DESCRIPTION

Various embodiments of the invention are described herein as follows. In an illustrative aspect, a contrast agent composition is provided. The contrast agent composition comprises a plurality of nanoparticles. As used herein, the terms “nanoparticles” and “nanostructures” are used synonymously to refer to particles having diameters in all dimensions of greater than 1 nm and less than 1 μm. Nanoparticles are typically spherical or substantially spherical. Nanostrutures may also be cube-shaped, cylindrical, star-shaped, or any other useful shape. All embodiments herein indicated as nanoparticles may also encompass nanostructures (e.g., nanostars), or may be limited to spherical nanoparticles, unless otherwise indicated.

In an embodiment, one or more nanoparticles comprises a magnetic nanoparticle. In an embodiment, the magnetic nanoparticle is selected from the group consisting of a ferromagnetic nanoparticle, a ferrimagnetic nanoparticle, a paramagnetic nanoparticle, a superparamagnetic nanoparticle, and any combination thereof. In an embodiment, the magnetic nanoparticle comprises a ferromagnetic nanoparticle. In an embodiment, the magnetic nanoparticle comprises a ferrimagnetic nanoparticle. In an embodiment, the magnetic nanoparticle comprises a paramagnetic nanoparticle. In an embodiment, the magnetic nanoparticle comprises a superparamagnetic nanoparticle.

In an embodiment, one or more nanoparticles comprises an iron nanoparticle. In an embodiment, the iron nanoparticle comprises iron nitride, iron oxide, or a combination thereof. In an embodiment, the iron nanoparticle comprises iron oxide. In an embodiment, the iron nanoparticle comprises iron nitride.

In an embodiment, the iron nitride comprises at least one phase of iron nitride selected from the group consisting of α-FeN (α expanded body-centered cubic (bcc)-Fe), α″-Fe₁₆N₂ (alpha double prime iron 16, also referred to as Fe₈N), γ′-Fe₄N, ϵ-Fe₃₋ₓN (0≤x≤1), ζ-Fe₂N, γ″-FeN, γ′″-FeN, and any combination thereof.

In an embodiment, the iron nitride comprises Fe₁₆N₂. In an embodiment, the iron nitride comprises Fe₄N. In an embodiment, the iron nitride comprises α-FeN. In an embodiment, the iron nitride comprises α″-Fe₁₆N₂. In an embodiment, the iron nitride comprises γ′-Fe₄N. In an embodiment, the iron nitride comprises ϵ-Fe₃₋ₓN (0≤x≤1). In an embodiment, the iron nitride comprises ζ-Fe₂N. In an embodiment, the iron nitride comprises γ″-FeN. In an embodiment, the iron nitride comprises γ′″-FeN.

Low nitrogen content phases (e.g., as γ-Fe₄N, ϵ-Fe₂₋₃N, α′-Fe₈N, and α″-Fe₁₆N₂) are ferromagnetic compounds that have well-characterized stoichiometry and electronic properties, thus rendering them attractive compounds for magnetic functional nanomaterials. The Fe₁₆N₂ phase is ferromagnetic above the single-domain size limit, indicating that it possesses an array of atomic moments exhibiting very strong interactions. Ferromagnetic materials exhibit parallel alignment of moments resulting in large net magnetization, even in the absence of a magnetic field. Fe₁₆N₂, in particular the α″-Fe₁₆N₂ phase, is a material of interest for many applications due to its exceptionally high magnetic moment which is larger than α-iron.

In an embodiment, the iron nanoparticle comprises i) a core comprising Fe₁₆N₂ and ii) a shell on the surface of the nanoparticle. In an embodiment, the shell comprises Fe, FeO, or a combination thereof. In an embodiment, the shell comprises Fe. In an embodiment, the shell comprises FeO.

In an embodiment, the plurality of nanoparticles has an average diameter between 1 nm to 999 nm. In an embodiment, the plurality of nanoparticles has an average diameter between 1 nm to 500 nm. In an embodiment, the plurality of nanoparticles has an average diameter between 1 nm to 400 nm. In an embodiment, the plurality of nanoparticles has an average diameter between 1 nm to 300 nm. In an embodiment, the plurality of nanoparticles has an average diameter between 1 nm to 200 nm. In an embodiment, the plurality of nanoparticles has an average diameter between 100 nm to 200 nm. In an embodiment, the plurality of nanoparticles has an average diameter between 1 nm to 100 nm. In an embodiment, the plurality of nanoparticles has an average diameter between 10 nm to 90 nm. In an embodiment, the plurality of nanoparticles has an average diameter between 20 nm to 80 nm. In an embodiment, the plurality of nanoparticles has an average diameter between 35 nm to 75 nm.

In an embodiment, one or more nanoparticles comprises one or more substituents on the surface of the nanoparticle. In an embodiment, the substituent comprises an organic substituent. In an embodiment, the organic substituent comprises a saccharide. In an embodiment, the organic substituent comprises a polysaccharide. In an embodiment, the organic substituent comprises a poly(xy(substituted or unsubstituted (C2-C3)alkyl)). In an embodiment, the organic substituent comprises a substituted or unsubstituted (Cᵢ-C₂oo)hydrocarbyl group interrupted by 0, 1, 2, or 3 groups independently chosen from -0-, —S—, -(0(C2-C3)alkylene)ₙ- In an embodiment, n is 1 to 1,000. In an embodiment, the organic substituent comprises a substituted —NH—. In an embodiment, the organic substituent comprises an unsubstituted —NH—.

In an embodiment, the organic substituent is selected from the group consisting of alginate, chitosan, curdlan, dextran, derivatized dextran, emulsan, a galactoglucopolysaccharide, geiian, glucuronan, N-acetyl-glucosamine, N-acetyl-heparosan, hyaluronic acid, kefiran, ientinan, levan, mauran, pullulan, scleroglucan, schizophyllan, stewartan, succinoglycan, xanthan, diutan, welan, starch, derivatized starch, tamarind, tragacanth, guar gum, derivatized guar gum, gum ghatti, gum arable, locust bean gum, cellulose, and derivatized cellulose, and any combination thereof. In an embodiment, the organic substituent is selected from the group consisting of alginate, polyethyleneglycol, polyethyleneglycol-COOH, and any combination thereof.

In an embodiment, the substituent comprises an inorganic substituent. In an embodiment, the inorganic substituent is selected from the group consisting of a metal, a halide, a lanthanide, a non-carbon molecule, and any combination thereof.

In an embodiment, the substituent comprises a silicon-based substituent. In an embodiment, the silicon-based substituent comprises SiO₂. In an embodiment, the silicon-based substituent comprises a Si-based polymer. In an embodiment, the silicon-based substituent comprises a Si-substituted monomer. In an embodiment, the silicon-based substituent comprises a Si-substituted polymer.

In an embodiment, the substituent comprises a drug. In an embodiment, the substituent comprises an antibody. In an embodiment, the substituent comprises a molecule for targeting a biomarker. In an embodiment, the substituent comprises a biological moiety. In an embodiment, the substituent comprises a fluorescent molecule for tracking.

In an embodiment, the one or more substituents on the surface of the nanoparticle comprise a number of substituents between 1 substituent and 10,000,000 substituents. In an embodiment, the one or more substituents on the surface of the nanoparticle are crosslinked. In an embodiment, the crosslinked substituents comprise direct crosslinking between the substituents. In an embodiment, the crosslinked substituents comprise crosslinking between the substituents via one or more linkers. In an embodiment, the one or more substituents on the surface of the nanoparticle are crosslinked, and in an embodiment, one of the crosslinked substituents comprises a drug, a fluorescent molecule, a protein, an antibody, or a biomarker. In an embodiment, the crosslinking comprises EDC/sulfo-NHS crosslinking. In an embodiment, the crosslinking comprises crosslinking via functional groups. In an embodiment, the crosslinking comprises crosslinking via electrostatics. In an embodiment, the crosslinking comprises crosslinking via adsorption.

In an embodiment, the contrast agent composition is a powder. In an embodiment, the contrast agent composition is lyophilized.

In an embodiment, the contrast agent composition further comprises a liquid. In an embodiment, the liquid is a medium acceptable for use in magnetic resonance imaging (MRI). In an embodiment, the liquid is saline. In an embodiment, the liquid is a sterile fluid.

In an embodiment, the liquid is water. In an embodiment, the contrast agent composition is a solution. In an embodiment, the contrast agent composition is a suspension.

In an embodiment, the contrast agent composition has a concentration of nanoparticles between 0.0001 μg/mL to 1 g/mL. In an embodiment, the contrast agent composition has a concentration of nanoparticles of 0.0001 ug/mL. In an embodiment, the contrast agent composition has a concentration of nanoparticles of 0.001 μg/mL. In an embodiment, the contrast agent composition has a concentration of nanoparticles of 0.01 ug/mL. In an embodiment, the contrast agent composition has a concentration of nanoparticles of 0.1 ug/mL. In an embodiment, the contrast agent composition has a concentration of nanoparticles of 1 ug/mL. In an embodiment, the contrast agent composition has a concentration of nanoparticles of 0.01 mg/mL. In an embodiment, the contrast agent composition has a concentration of nanoparticles of 0.1 mg/mL. In an embodiment, the contrast agent composition has a concentration of nanoparticles of 1 mg/mL. In an embodiment, the contrast agent composition has a concentration of nanoparticles of 0.01 g/mL. In an embodiment, the contrast agent composition has a concentration of nanoparticles of 0.1 g/mL. In an embodiment, the contrast agent composition has a concentration of nanoparticles of 1 g/mL. In an embodiment, the contrast agent composition has a concentration of nanoparticles greater than 0.0001 ug/mL. In an embodiment, the contrast agent composition has a concentration of nanoparticles greater than 0.001 μg/mL. In an embodiment, the contrast agent composition has a concentration of nanoparticles greater than 0.01 ug/mL. In an embodiment, the contrast agent composition has a concentration of nanoparticles greater than 0.1 ug/mL. In an embodiment, the contrast agent composition has a concentration of nanoparticles greater than 1 ug/mL. In an embodiment, the contrast agent composition has a concentration of nanoparticles greater than 0.01 mg/mL. In an embodiment, the contrast agent composition has a concentration of nanoparticles greater than 0.1 mg/mL. In an embodiment, the contrast agent composition has a concentration of nanoparticles greater than 1 mg/mL. In an embodiment, the contrast agent composition has a concentration of nanoparticles greater than 0.01 g/mL. In an embodiment, the contrast agent composition has a concentration of nanoparticles greater than 0.1 g/mL. In an embodiment, the contrast agent composition has a concentration of nanoparticles greater than 1 g/mL.

In an embodiment, the contrast agent composition has a concentration of nanoparticles less than 0.0001 ug/mL. In an embodiment, the contrast agent composition has a concentration of nanoparticles less than 0.001 μg/mL. In an embodiment, the contrast agent composition has a concentration of nanoparticles less than 0.01 ug/mL. In an embodiment, the contrast agent composition has a concentration of nanoparticles less than 0.1 ug/mL. In an embodiment, the contrast agent composition has a concentration of nanoparticles less than 1 ug/mL. In an embodiment, the contrast agent composition has a concentration of nanoparticles less than 0.01 mg/mL. In an embodiment, the contrast agent composition has a concentration of nanoparticles less than 0.1 mg/mL. In an embodiment, the contrast agent composition has a concentration of nanoparticles less than 1 mg/mL. In an embodiment, the contrast agent composition has a concentration of nanoparticles less than 0.01 g/mL. In an embodiment, the contrast agent composition has a concentration of nanoparticles less than 0.1 g/mL. In an embodiment, the contrast agent composition has a concentration of nanoparticles less than 1 g/mL.

In an embodiment, the contrast agent composition is configured for injection in a subject. In an embodiment, the contrast agent composition is configured for targeting cancer in a subject. In an embodiment, the contrast agent composition is configured for targeting a biomarker in a subject. In an embodiment, the contrast agent composition is configured for magnetic resonance imaging (MRI) of a sample.

In an illustrative aspect, a method for magnetic resonance imaging of a contrast agent composition is provided. The method comprises i) scanning a region of a subject having the contrast agent composition; ii) detecting the contrast agent composition, and iii) generating a contrast agent image. Magnetic resonance imaging (MRI) is a powerful imaging technique that uses a magnetic field and radio waves to create high resolution images of the organs and tissues of the body. In operation, the magnetic field produced by an MRI machine realigns hydrogen atoms in the body. Radio waves cause the aligned atoms to produce very faint signals, which are then used to create cross-sectional MRI images. MRI machines can also be used to produce 3-D images of organs and other tissues. MRI provides a noninvasive examination tool that is used widely to diagnose a variety of problems and has proven to be a powerful research tool in the medical field. The contrast agent composition described herein are administered according to the described methods to increase the visibility of internal body structures when using MRI.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

In some embodiments, methods comprising administering a contrast agent composition described herein to a subject, tissue, sample, or cell. In some embodiments, the contrast agent composition is administered locally to a site of treatment, diagnosis, or observation. In some embodiments, the contrast agent composition is administered systemically.

As used herein, “detecting” includes the use of both the instrument used (if used) to observe and record a signal, the reagents required to generate that signal, and/or analysis of signals to generate an image or value. In various embodiments, a level is detected using any suitable method, including magnetic resonance imaging, fluorescence, microscopy, other imagine techniques (e.g., CT, PET, SPECT, etc.), and the like.

“Diagnose”, “diagnosing”, “diagnosis”, and variations thereof refer to the detection, determination, or recognition of a health status or condition of a subject on the basis of one or more signs, symptoms, data, or other information pertaining to that individual. The health status of a subject can be diagnosed as healthy/normal (e.g., a diagnosis of the absence of a disease or condition) or diagnosed as ill/abnormal (e.g., a diagnosis of the presence of a disease or condition, etc.). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual.

“Prognose”, “prognosing”, “prognosis”, and variations thereof refer to the prediction of a future course of a disease or condition in a subject who has the disease or condition (e.g., predicting patient survival, predicting the need for interventions, predicting the aggressiveness of a disease, predicting the responsiveness of a disease to a particular treatment, etc.), and such terms encompass the evaluation of disease response after the administration of a treatment or therapy to the subject. Example prognoses include likelihood of mortality (e.g., <1%, <5%, <10<, <20%, <30%, <40%, <50%, >50%, >60%, >70%, >80%, >90%, >95%, >99%), likelihood of responsiveness/resistance to treatment (e.g., <1%, <5%, <10<, <20%, <30%, <40%, <50%, >50%, >60%, >70%, >80%, >90%, >95%, >99%), likely lifespan (e.g., <1 month, <2 months, <3 month, <6 months, <1 year, 2 years, 3 years, >3 years, etc.).

In an illustrative aspect, a method of performing magnetic resonance imaging is provided. The method comprises i) providing a contrast agent composition of dispersed in a medium; ii) illuminating the contrast agent composition with an excitatory electromagnetic pulse; and iii) detecting electromagnetic radiation emitted from the contrast agent composition with a detection system. The previously described embodiments of the method for magnetic resonance imaging of a contrast agent composition are applicable to the method of performing magnetic resonance imaging described herein.

In an illustrative aspect, a kit is provided. The kit comprises i) a contrast agent composition and ii) a liquid. The previously described embodiments of the contrast agent composition are applicable to the kit described herein.

In an embodiment, the liquid comprises at least one pharmaceutically acceptable carrier. The pharmaceutically acceptable carrier can be, for example, a diluent or other excipient including at least one of water like water for injection, saline, dextrose, glycerol, or the like, and any combination thereof. A pharmaceutically acceptable carrier may contain wetting or emulsifying agents, stabilizing or pH-buffering agents, and the like.

In an embodiment, the liquid comprises a further ingredient. In an embodiment, the further ingredient comprises a targeting moiety. In an embodiment, the targeting moiety is coupled to the contrast agent composition. In an embodiment, the targeting moiety is selected from the group consisting of a protein, an enzyme, a peptide, an antibody, and any combination thereof.

The following numbered embodiments are contemplated and are non-limiting:

1. A contrast agent composition comprising a plurality of nanoparticles.
2. The contrast agent composition of clause 1, any other suitable clause, or any combination of suitable clauses, wherein one or more nanoparticles comprises a magnetic nanoparticle.
3. The contrast agent composition of clause 2, any other suitable clause, or any combination of suitable clauses, wherein the magnetic nanoparticle is selected from the group consisting of a ferromagnetic nanoparticle, a ferrimagnetic nanoparticle, a paramagnetic nanoparticle, a superparamagnetic nanoparticle, and any combination thereof.
4. The contrast agent composition of clause 2, any other suitable clause, or any combination of suitable clauses, wherein the magnetic nanoparticle comprises a ferromagnetic nanoparticle.
5. The contrast agent composition of clause 2, any other suitable clause, or any combination of suitable clauses, wherein the magnetic nanoparticle comprises a ferrimagnetic nanoparticle.
6. The contrast agent composition of clause 2, any other suitable clause, or any combination of suitable clauses, wherein the magnetic nanoparticle comprises a paramagnetic nanoparticle.
7. The contrast agent composition of clause 2, any other suitable clause, or any combination of suitable clauses, wherein the magnetic nanoparticle comprises a superparamagnetic nanoparticle.
8. The contrast agent composition of clause 1, any other suitable clause, or any combination of suitable clauses, wherein one or more nanoparticles comprises an iron nanoparticle.
9. The contrast agent composition of clause 8, any other suitable clause, or any combination of suitable clauses, wherein the iron nanoparticle comprises iron nitride, iron oxide, or a combination thereof.
10. The contrast agent composition of clause 8, any other suitable clause, or any combination of suitable clauses, wherein the iron nanoparticle comprises iron oxide.
11. The contrast agent composition of clause 8, any other suitable clause, or any combination of suitable clauses, wherein the iron nanoparticle comprises iron nitride.
12. The contrast agent composition of clause 11, any other suitable clause, or any combination of suitable clauses, wherein the iron nitride comprises at least one phase of iron nitride selected from the group consisting of α-FeN (α expanded body-centered cubic (bcc)-Fe), α″-Fe₁₆N₂ (alpha double prime iron 16, also referred to as Fe₈N), γ′-Fe₄N, ϵ-Fe₃₋ₓN (0≤x≤1), ζ-Fe₂N, γ″-FeN, γ′″-FeN, and any combination thereof.
13. The contrast agent composition of clause 11, any other suitable clause, or any combination of suitable clauses, wherein the iron nitride comprises Fe₁₆N₂.
14. The contrast agent composition of clause 11, any other suitable clause, or any combination of suitable clauses, wherein the iron nitride comprises Fe₄N.
15. The contrast agent composition of clause 11, any other suitable clause, or any combination of suitable clauses, wherein the iron nitride comprises α-FeN.
16. The contrast agent composition of clause 11, any other suitable clause, or any combination of suitable clauses, wherein the iron nitride comprises α″-Fe₁₆N₂.
17. The contrast agent composition of clause 11, any other suitable clause, or any combination of suitable clauses, wherein the iron nitride comprises γ′-Fe₄N.
18. The contrast agent composition of clause 11, any other suitable clause, or any combination of suitable clauses, wherein the iron nitride comprises ϵ-Fe₃₋ₓN (0≤x≤1).
19. The contrast agent composition of clause 11, any other suitable clause, or any combination of suitable clauses, wherein the iron nitride comprises ζ-Fe₂N.
20. The contrast agent composition of clause 11, any other suitable clause, or any combination of suitable clauses, wherein the iron nitride comprises γ″-FeN.
21. The contrast agent composition of clause 11, any other suitable clause, or any combination of suitable clauses, wherein the iron nitride comprises γ′″-FeN.
22. The contrast agent composition of clause 8, any other suitable clause, or any combination of suitable clauses, wherein the iron nanoparticle comprises i) a core comprising Fe₁₆N₂ and ii) a shell on the surface of the nanoparticle.
23. The contrast agent composition of clause 22, any other suitable clause, or any combination of suitable clauses, wherein the shell comprises Fe, FeO, or a combination thereof.
24. The contrast agent composition of clause 22, any other suitable clause, or any combination of suitable clauses, wherein the shell comprises Fe.
25. The contrast agent composition of clause 22, any other suitable clause, or any combination of suitable clauses, wherein the shell comprises FeO.
26. The contrast agent composition of clause 1, any other suitable clause, or any combination of suitable clauses, wherein the plurality of nanoparticles has an average diameter between 1 nm to 999 nm.
27. The contrast agent composition of clause 26, any other suitable clause, or any combination of suitable clauses, wherein the plurality of nanoparticles has an average diameter between 1 nm to 500 nm.
28. The contrast agent composition of clause 26, any other suitable clause, or any combination of suitable clauses, wherein the plurality of nanoparticles has an average diameter between 1 nm to 400 nm.
29. The contrast agent composition of clause 26, any other suitable clause, or any combination of suitable clauses, wherein the plurality of nanoparticles has an average diameter between 1 nm to 300 nm.
30. The contrast agent composition of clause 26, any other suitable clause, or any combination of suitable clauses, wherein the plurality of nanoparticles has an average diameter between 1 nm to 200 nm.
31. The contrast agent composition of clause 26, any other suitable clause, or any combination of suitable clauses, wherein the plurality of nanoparticles has an average diameter between 100 nm to 200 nm.
32. The contrast agent composition of clause 26, any other suitable clause, or any combination of suitable clauses, wherein the plurality of nanoparticles has an average diameter between 1 nm to 100 nm.
33. The contrast agent composition of clause 26, any other suitable clause, or any combination of suitable clauses, wherein the plurality of nanoparticles has an average diameter between 10 nm to 90 nm.
34. The contrast agent composition of clause 26, any other suitable clause, or any combination of suitable clauses, wherein the plurality of nanoparticles has an average diameter between 20 nm to 80 nm.
35. The contrast agent composition of clause 26, any other suitable clause, or any combination of suitable clauses, wherein the plurality of nanoparticles has an average diameter between 35 nm to 75 nm.
36. The contrast agent composition of clause 1, any other suitable clause, or any combination of suitable clauses, wherein one or more nanoparticles comprises one or more substituents on the surface of the nanoparticle.
37. The contrast agent composition of clause 36, any other suitable clause, or any combination of suitable clauses, wherein the substituent comprises an organic substituent.
38. The contrast agent composition of clause 37, any other suitable clause, or any combination of suitable clauses, wherein the organic substituent comprises a saccharide.
39. The contrast agent composition of clause 37, any other suitable clause, or any combination of suitable clauses, wherein the organic substituent comprises a polysaccharide.
40. The contrast agent composition of clause 37, any other suitable clause, or any combination of suitable clauses, wherein the organic substituent comprises a poly(xy(substituted or unsubstituted (C2-C3)alkyl)).
41. The contrast agent composition of clause 37, any other suitable clause, or any combination of suitable clauses, wherein the organic substituent comprises a substituted or unsubstituted (Cᵢ-C₂oo)hydrocarbyl group interrupted by 0, 1, 2, or 3 groups independently chosen from -0-, —S—, -(0(C2-C3)alkylene)ₙ- wherein n is 1 to 1,000.
42. The contrast agent composition of clause 37, any other suitable clause, or any combination of suitable clauses, wherein the organic substituent comprises a substituted —NH—.
43. The contrast agent composition of clause 37, any other suitable clause, or any combination of suitable clauses, wherein the organic substituent comprises an unsubstituted —NH—.
44. The contrast agent composition of clause 37, any other suitable clause, or any combination of suitable clauses, wherein the organic substituent is selected from the group consisting of alginate, chitosan, curdlan, dextran, derivatized dextran, emulsan, a galactoglucopolysaccharide, geiian, glucuronan, N-acetyl-glucosamine, N-acetyl-heparosan, hyaluronic acid, kefiran, ientinan, levan, mauran, pullulan, scleroglucan, schizophyllan, stewartan, succinoglycan, xanthan, diutan, welan, starch, derivatized starch, tamarind, tragacanth, guar gum, derivatized guar gum, gum ghatti, gum arable, locust bean gum, cellulose, and derivatized cellulose, and any combination thereof.
45. The contrast agent composition of clause 37, any other suitable clause, or any combination of suitable clauses, wherein the organic substituent is selected from the group consisting of alginate, polyethyleneglycol, polyethyleneglycol-COOH, and any combination thereof
46. The contrast agent composition of clause 36, any other suitable clause, or any combination of suitable clauses, wherein the substituent comprises an inorganic substituent.
47. The contrast agent composition of clause 46, any other suitable clause, or any combination of suitable clauses, wherein the inorganic substituent is selected from the group consisting of a metal, a halide, a lanthanide, a non-carbon molecule, and any combination thereof.
48. The contrast agent composition of clause 36, any other suitable clause, or any combination of suitable clauses, wherein the substituent comprises a silicon-based substituent.
49. The contrast agent composition of clause 48, any other suitable clause, or any combination of suitable clauses, wherein the silicon-based substituent comprises SiO₂.
50. The contrast agent composition of clause 48, any other suitable clause, or any combination of suitable clauses, wherein the silicon-based substituent comprises a Si-based polymer.
51. The contrast agent composition of clause 48, any other suitable clause, or any combination of suitable clauses, wherein the silicon-based substituent comprises a Si-substituated monomer
52. The contrast agent composition of clause 48, any other suitable clause, or any combination of suitable clauses, wherein the silicon-based substituent comprises a Si-substituated polymer.
53. The contrast agent composition of clause 36, any other suitable clause, or any combination of suitable clauses, wherein the substituent comprises a drug.
54. The contrast agent composition of clause 36, any other suitable clause, or any combination of suitable clauses, wherein the substituent comprises an antibody.
55. The contrast agent composition of clause 36, any other suitable clause, or any combination of suitable clauses, wherein the substituent comprises a molecule for targeting a biomarker
56. The contrast agent composition of clause 36, any other suitable clause, or any combination of suitable clauses, wherein the substituent comprises a biological moiety.
57. The contrast agent composition of clause 36, any other suitable clause, or any combination of suitable clauses, wherein the substituent comprises a fluorescent molecule for tracking.
58. The contrast agent composition of clause 36, any other suitable clause, or any combination of suitable clauses, wherein the one or more substituents on the surface of the nanoparticle comprise a number of substituents between 1 substituent and 10,000,000 substituents.
59. The contrast agent composition of clause 36, any other suitable clause, or any combination of suitable clauses, wherein the one or more substituents on the surface of the nanoparticle are crosslinked.
60. The contrast agent composition of clause 59, any other suitable clause, or any combination of suitable clauses, wherein the crosslinked substituents comprise direct crosslinking between the substituents.
61. The contrast agent composition of clause 59, any other suitable clause, or any combination of suitable clauses, wherein the crosslinked substituents comprise crosslinking between the substituents via one or more linkers.
62. The contrast agent composition of clause 36, any other suitable clause, or any combination of suitable clauses, wherein the one or more substituents on the surface of the nanoparticle are crosslinked, and wherein one of the crosslinked substituents comprises a drug, a fluorescent molecule, a protein, an antibody, or a biomarker.
63. The contrast agent composition of clause 62, any other suitable clause, or any combination of suitable clauses, wherein the crosslinking comprises EDC/sulfo-NHS crosslinking.
64. The contrast agent composition of clause 62, any other suitable clause, or any combination of suitable clauses, wherein the crosslinking comprises crosslinking via functional groups.
65. The contrast agent composition of clause 62, any other suitable clause, or any combination of suitable clauses, wherein the crosslinking comprises crosslinking via electrostatics.
66. The contrast agent composition of clause 62, any other suitable clause, or any combination of suitable clauses, wherein the crosslinking comprises crosslinking via adsorption.
67. The contrast agent composition of clause 1, any other suitable clause, or any combination of suitable clauses, wherein the contrast agent composition is a powder.
68. The contrast agent composition of clause 1, any other suitable clause, or any combination of suitable clauses, wherein the contrast agent composition is lyophilized.
69. The contrast agent composition of clause 1, any other suitable clause, or any combination of suitable clauses, wherein the contrast agent composition further comprises a liquid.
70. The contrast agent composition of clause 69, any other suitable clause, or any combination of suitable clauses, wherein the liquid is a medium acceptable for use in magnetic resonance imaging (MRI).
71. The contrast agent composition of clause 69, any other suitable clause, or any combination of suitable clauses, wherein the liquid is saline.
72. The contrast agent composition of clause 69, any other suitable clause, or any combination of suitable clauses, wherein the liquid is a sterile fluid.
73. The contrast agent composition of clause 69, any other suitable clause, or any combination of suitable clauses, wherein the liquid is water.
74. The contrast agent composition of clause 69, any other suitable clause, or any combination of suitable clauses, wherein the contrast agent composition is a solution.
75. The contrast agent composition of clause 69, any other suitable clause, or any combination of suitable clauses, wherein the contrast agent composition is a suspension.
76. The contrast agent composition of clause 69, any other suitable clause, or any combination of suitable clauses, wherein the contrast agent composition has a concentration of nanoparticles between 0.0001 μg/mL to 1 g/mL
77. The contrast agent composition of clause 69, any other suitable clause, or any combination of suitable clauses, wherein the contrast agent composition has a concentration of nanoparticles of 0.0001 ug/mL.
78. The contrast agent composition of clause 69, any other suitable clause, or any combination of suitable clauses, wherein the contrast agent composition has a concentration of nanoparticles of 0.001 μg/mL.
79. The contrast agent composition of clause 69, any other suitable clause, or any combination of suitable clauses, wherein the contrast agent composition has a concentration of nanoparticles of 0.01 ug/mL.
80. The contrast agent composition of clause 69, any other suitable clause, or any combination of suitable clauses, wherein the contrast agent composition has a concentration of nanoparticles of 0.1 ug/mL.
81. The contrast agent composition of clause 69, any other suitable clause, or any combination of suitable clauses, wherein the contrast agent composition has a concentration of nanoparticles of 1 ug/mL.
82. The contrast agent composition of clause 69, any other suitable clause, or any combination of suitable clauses, wherein the contrast agent composition has a concentration of nanoparticles of 0.01 mg/mL.
83. The contrast agent composition of clause 69, any other suitable clause, or any combination of suitable clauses, wherein the contrast agent composition has a concentration of nanoparticles of 0.1 mg/mL.
84. The contrast agent composition of clause 69, any other suitable clause, or any combination of suitable clauses, wherein the contrast agent composition has a concentration of nanoparticles of 1 mg/mL.
85. The contrast agent composition of clause 69, any other suitable clause, or any combination of suitable clauses, wherein the contrast agent composition has a concentration of nanoparticles of 0.01 g/mL.
86. The contrast agent composition of clause 69, any other suitable clause, or any combination of suitable clauses, wherein the contrast agent composition has a concentration of nanoparticles of 0.1 g/mL.
87. The contrast agent composition of clause 69, any other suitable clause, or any combination of suitable clauses, wherein the contrast agent composition has a concentration of nanoparticles of 1 g/mL.
88. The contrast agent composition of clause 69, any other suitable clause, or any combination of suitable clauses, wherein the contrast agent composition has a concentration of nanoparticles greater than 0.0001 ug/mL.
89. The contrast agent composition of clause 69, any other suitable clause, or any combination of suitable clauses, wherein the contrast agent composition has a concentration of nanoparticles greater than 0.001 μg/mL.
90. The contrast agent composition of clause 69, any other suitable clause, or any combination of suitable clauses, wherein the contrast agent composition has a concentration of nanoparticles greater than 0.01 ug/mL.
91. The contrast agent composition of clause 69, any other suitable clause, or any combination of suitable clauses, wherein the contrast agent composition has a concentration of nanoparticles greater than 0.1 ug/mL.
92. The contrast agent composition of clause 69, any other suitable clause, or any combination of suitable clauses, wherein the contrast agent composition has a concentration of nanoparticles greater than 1 ug/mL.
93. The contrast agent composition of clause 69, any other suitable clause, or any combination of suitable clauses, wherein the contrast agent composition has a concentration of nanoparticles greater than 0.01 mg/mL.
94. The contrast agent composition of clause 69, any other suitable clause, or any combination of suitable clauses, wherein the contrast agent composition has a concentration of nanoparticles greater than 0.1 mg/mL.
95. The contrast agent composition of clause 69, any other suitable clause, or any combination of suitable clauses, wherein the contrast agent composition has a concentration of nanoparticles greater than 1 mg/mL.
96. The contrast agent composition of clause 69, any other suitable clause, or any combination of suitable clauses, wherein the contrast agent composition has a concentration of nanoparticles greater than 0.01 g/mL.
97. The contrast agent composition of clause 69, any other suitable clause, or any combination of suitable clauses, wherein the contrast agent composition has a concentration of nanoparticles greater than 0.1 g/mL.
98. The contrast agent composition of clause 69, any other suitable clause, or any combination of suitable clauses, wherein the contrast agent composition has a concentration of nanoparticles greater than 1 g/mL.
99. The contrast agent composition of clause 69, any other suitable clause, or any combination of suitable clauses, wherein the contrast agent composition has a concentration of nanoparticles less than 0.0001 ug/mL.
100. The contrast agent composition of clause 69, any other suitable clause, or any combination of suitable clauses, wherein the contrast agent composition has a concentration of nanoparticles less than 0.001 μg/mL.
101. The contrast agent composition of clause 69, any other suitable clause, or any combination of suitable clauses, wherein the contrast agent composition has a concentration of nanoparticles less than 0.01 ug/mL.
102. The contrast agent composition of clause 69, any other suitable clause, or any combination of suitable clauses, wherein the contrast agent composition has a concentration of nanoparticles less than 0.1 ug/mL.
103. The contrast agent composition of clause 69, any other suitable clause, or any combination of suitable clauses, wherein the contrast agent composition has a concentration of nanoparticles less than 1 ug/mL.
104. The contrast agent composition of clause 69, any other suitable clause, or any combination of suitable clauses, wherein the contrast agent composition has a concentration of nanoparticles less than 0.01 mg/mL.
105. The contrast agent composition of clause 69, any other suitable clause, or any combination of suitable clauses, wherein the contrast agent composition has a concentration of nanoparticles less than 0.1 mg/mL.
106. The contrast agent composition of clause 69, any other suitable clause, or any combination of suitable clauses, wherein the contrast agent composition has a concentration of nanoparticles less than 1 mg/mL.
107. The contrast agent composition of clause 69, any other suitable clause, or any combination of suitable clauses, wherein the contrast agent composition has a concentration of nanoparticles less than 0.01 g/mL.
108. The contrast agent composition of clause 69, any other suitable clause, or any combination of suitable clauses, wherein the contrast agent composition has a concentration of nanoparticles less than 0.1 g/mL.
109. The contrast agent composition of clause 69, any other suitable clause, or any combination of suitable clauses, wherein the contrast agent composition has a concentration of nanoparticles less than 1 g/mL.
110. The contrast agent composition of clause 1, any other suitable clause, or any combination of suitable clauses, wherein the contrast agent composition is configured for injection in a subject.
111. The contrast agent composition of clause 1, any other suitable clause, or any combination of suitable clauses, wherein the contrast agent composition is configured for targeting cancer in a subject.
112. The contrast agent composition of clause 1, any other suitable clause, or any combination of suitable clauses, wherein the contrast agent composition is configured for targeting a biomarker in a subject.
113. The contrast agent composition of clause 1, any other suitable clause, or any combination of suitable clauses, wherein the contrast agent composition is configured for magnetic resonance imaging (MRI) of a sample.
114. A method for magnetic resonance imaging of a contrast agent composition, the method comprising:

i. scanning a region of a subject having the contrast agent composition;

ii. detecting the contrast agent composition, and

iii. generating a contrast agent image.

115. The method of clause 114, wherein the contrast agent composition is the contrast agent composition of any of clauses 1 to 113.
116. The method of clause 114, wherein generating the contrast agent imaging comprises generating an image of the contrast agent on a display.
117. A method of performing magnetic resonance imaging, the method comprising:

i. providing the contrast agent composition of clause 1 dispersed in a medium;

ii. illuminating the contrast agent composition with an excitatory electromagnetic pulse; and

iii. detecting electromagnetic radiation emitted from the contrast agent composition with a detection system.

118. The method of clause 114, wherein the contrast agent composition is the contrast agent composition of any of clauses 1 to 116.
119. The method of clause 114, wherein the detection system generates an image of the contrast agent composition on a display.
120. A kit comprising i) a contrast agent composition and ii) a liquid.
121. The kit of clause 120, wherein the contrast agent composition is the contrast agent composition of any of clauses 1 to 113.
122. The kit of clause 120, wherein the liquid comprises at least one pharmaceutically acceptable carrier.
123. The kit of clause 120, wherein the liquid comprises a further ingredient.
124. The kit of clause 123, wherein the further ingredient comprises a targeting moiety.
125. The kit of clause 124, wherein the targeting moiety is coupled to the contrast agent composition.
126. The kit of clause 124, wherein the targeting moiety is selected from the group consisting of a protein, an enzyme, a peptide, an antibody, and any combination thereof.

### EXAMPLE 1

### Synthesis of Nanoparticles for Contrast Agent Compositions

In the instant example, exemplary nanoparticles comprising Fe₁₆N₂ were prepared. Iron nitride nanoparticles were synthesized via solvothermal and solid-gas phase reaction, in which iron oxide powder was intermediate.

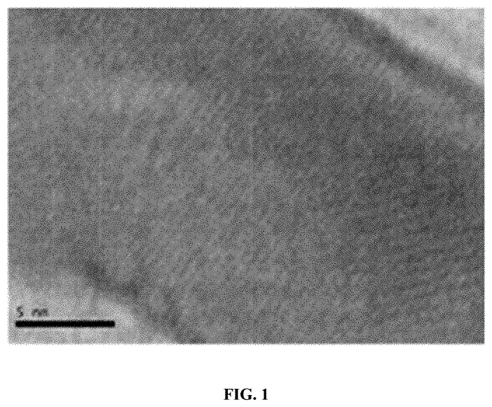

The solvothermal method is performed in organic solvents at (60-750)° C. before being converted to iron nitride. This results in excellent crystallinity, as demonstrated in the HRTEM image of FIG. 1. High relaxivity materials can be obtained from higher iron concentrations and better crystalline structures (e.g., low or no defect states). According to the quantum mechanical outer sphere theory, T2 relaxivity is proportional to the Mₛ (magnetic saturation value) and the superparamagnetic particle radius. The relaxivity (r₂) in the motional average regime, assuming a spherical particle, is shown as:

r₂=(256π²γ²/405)κM_{S}²r²/D(1+L/r)=1/T2 (Eq. 1)

where γ is the proton gyromagnetic ratio; Mₛ and r are the saturation magnetization and effective radius of the magnetic nanoparticles, respectively; D is the diffusivity of water molecules; L is the thickness of an impermeable surface coating; and κ is the conversion factor (κ=V*=C_{Fe}, V* is the volume fraction, C_{Fe} is the concentration of Fe).

The structure of the nanoparticle composition was characterized using X-ray diffraction (XRD). The saturation magnetization (Msat) and coercivity of nanoparticles were determined using a superconducting quantum interference device (SQUID). Without being bound by any theory, results suggest that formation of the Fe₁₆N₂ phase may depend on temperature and selection of reducing agent. It is noted that Fe₁₆N₂ exhibits saturation magnetizations larger than that of α-Fe. Because many phases of iron nitride can show peaks at the same angle in XRD, SQUID magnetometry is an important tool phase identification. The highly magnetic Fe₁₆N₂ phase of iron nitride may be produced in high yields having good resistance to oxidation, exceptionally high blocking temperatures. This material has a high magnetic moment and advantageously does not contain costly rare earth elements or toxic cobalt. Additionally, the synthesis procedure produces minimal toxic waste byproduct.

### EXAMPLE 2

### Synthesis of Nanoparticles for Contrast Agent Compositions

In the instant example, exemplary nanoparticles comprising Fe₁₆N₂ can be prepared. Synthesis can be performed using a five step procedure: synthesis of an iron oleate precursor complex, synthesis of iron oxide nanoparticles, oleic acid cap removal and purification of iron oxide nanoparticles, reduction to α-iron, and nitrogenation under ammonia gas.

The precursor of the nanoparticles in the instant example is iron oleate, (iron(II,III) [(9Z)-9-octadecenoate]n) where n is the coordination number of iron and could form a monomer, dimer, or trimer. Iron oleate can be produced as known in the art and the iron oleate complex can be formed from the combination of sodium oleate salt (sodium (9Z)-9-octadecenoate) and iron(III) chloride hexahydrate (FeCl3·6H2O). The precursor can be further purified via washing with ethanol, acetone, hexane, and/or water and then dried in the oven. Subsequently, iron oxide nanoparticles can be prepared using a modified procedure as known in the art (see, e.g., Park et al., “Ultra-large-scale syntheses of monodisperse nanocrystals,” Nature Materials, 2004, 3: 891-895; herein incorporated by reference).

After synthesis, the iron oxide nanoparticles are capped with oleic acid. The cap can be removed and the iron oxide nanoparticles can be a powder sample reduced under UHP hydrogen gas. Accordingly, the zero-valent iron using a hydrogen gas reduction can be generated. The iron nitride nanoparticles can be produced using the zero-valent iron nanoparticles as a precursor.

### EXAMPLE 3

### Characterization of Nanoparticles for Contrast Agent Compositions

In the instant example, exemplary nanoparticles comprising Fe₁₆N₂ were characterized. In the entire temperature range up to 350 K, the Fe₁₆N₂ nanoparticles demonstrated strong ferromagnetic behavior as evidenced by the gap between the ZFC and FC curves persisting even at 350 K. From the ZFC curve, TB was estimated to be ˜350 K, but even above this temperature, the equilibrium magnetization of the nanoparticles was not reached. Superparamagnetic behavior of the nanoparticles was observed in this sample, as opposed to the larger samples (>20 nm), which demonstrate ferromagnetism.

The strong superparamagnetic behavior of the Fe₁₆N₂ nanoparticles was confirmed in magnetic hysteresis measurements. Consistent with the results of DC magnetization measurements, magnetic hysteresis measurements at 293 K performed on Fe₁₆N₂ nanocrystals did not demonstrate coercivity, thus verifying that the magnetic hyperthermia results from a Néel process.

Although an Msat value was unable to be identified with the field strengths presently available, extrapolating the line provides an estimate of Msat to be ˜100 emu/g. The DC (τm=100 s) magnetization of the nanoparticles was measured with a DC field of 100 Oe in the temperature range between 9 K and 350 K using a Quantum Design™ magnetic property measurement system (MPMS) superconducting quantum interference device (SQUID) magnetometer.

Measurements of the frequency-dependent volume susceptibility in the frequency range 1 Hz to 100 kHz were performed using a DynoMag® instrument (IMEGO AB, Sweden), with a frequency range from 1 Hz to 200 kHz, a resolution magnetic moment of 3×10-11 Am2, and an excitation amplitude of 0.5 mT. The nanoparticles in a water solvent at a concentration of 130 M was measured using a 200 μL sample. Measurements were performed on a sample of “base” ferrofluid (e.g., colloidal suspensions of either Fe₁₆N₂ or magnetite (Fe₃O₄) particles) having spherical morphology of mean particle diameter 15-18 nm in a deionized water solvent, with succinylated PEG as a capping agent.

Susceptometry data verifies the magnetic hysteresis measurement determining that the nanoparticles of <20 nm are superparamagnetic at room temperature. The susceptometry measurements demonstrate a single peak attributed to a Néel process in which τ_{N}=1.29×10⁻⁶ ms.

The Néel relaxation time of moment rotations activated by thermal fluctuation may be expressed as: τ_{N}=τ0 exp (KuV/kBT), where τ₀ is on the order of 10⁻⁹ s, V is the particle volume, k_{B} is the Boltzmann constant and Kᵤ is an effective anisotropy energy barrier. For iron oxide V=1.767×10⁻²⁴ m³. When k_{B}T>KᵤV, the magnetic moment flips at a measured time, demonstrating zero coercivity. Presently, the effective anisotropy energy (Kᵤ) of nanoparticles comprising iron oxide are estimated to be 4.2×10⁵ ergs/cc by the relation KᵤV=25k_{B}T_{B} (assuming T_{B}=215 K), 30 higher than the Kᵤ of bulk Fe₃O₄ (Kᵤ=6.4×104) due to additional anisotropies. The effective anisotropy energy of the nanoparticles comprising iron nitride was calculated to be 5.6×105ergs/cc. A reference value for bulk Fe₁₆N₂ is not presently available in the literature. The real part of the susceptibility (χ′) values for both samples is greater than zero, corresponding to a typical feature of ferri/ferromagnetic materials. Despite this, the χ′ value for nanoparticles comprising iron nitride is two times higher than the the χ′ value for nanoparticles comprising iron oxide. As expected, the real part of the susceptibility (χ′) curve remains above zero for both materials, corresponding to a typical feature of ferri/ferromagnetic materials.

### EXAMPLE 4

### Characterization of Nanoparticles for Contrast Agent Compositions

In the instant example, exemplary nanoparticles comprising Fe₁₆N₂ can be characterized. Nanoparticles can be characterized by X-Ray diffractometer (XRD), Transmission electron microscopy (TEM), and/or superconducting quantum interference device (SQUID) magnetometry.

For structural characterization, nanoparticles samples can be prepared by placing a drop of colloidal solution onto a 200-mesh carbon-coated copper grid. The sample can be fixed on the grid once the solvent evaporates away. A JEOL-2010F TEM equipped with an energy dispersive spectroscopy (EDS) apparatus can be used to determine the elemental composition of the nanoparticles. The electron beam can focus on a single nanoparticle and the characteristic X-ray peaks specific to each element can be identified.

The phase and crystal structure of the nanoparticles can be determined using an XRD with a Cu Ka source (0.154 nm) and attached monochromator. In preliminary studies, both the XRD and the TEM demonstrate a body-centered tetragonal (BCT) crystal system. This system is expected for Fe₁₆N₂ and can differentiate this iron nitride from iron or iron oxide. Using the library, peaks can be matched to peaks determined in preliminary work which correspond to the iron nitride, ICCD Card Nos. 10-070-6150 and 01-078-1865,for Fe₈N and Fe₁₆N₂, respectively.

Magnetic characterization of Fe₁₆N₂ has been performed using SQUID magnetometry and compared to iron oxide nanoparticles of similar size (˜18 nm). SQUID can be used to measure nanoparticles and a full hysteresis loop can be run on each nanoparticle sample at room temperature. The temperature dependence of magnetization for the Fe₁₆N₂ nanoparticle samples can be measured under zero-field cooled and field- cooled (ZFC and FC) conditions. The DC (τm=100 s) magnetization of the nanoparticle samples can be measured with a DC field of 100 Oe in the temperature range between 10K and 350K. Magnetic saturation (Mₛₐₜ) values for nanoparticles comprising iron nitride range from 787 to 2000 electromagnetic units/cm³ those for iron oxide range from 80 to 100 electromagnetic units/g.

### EXAMPLE 5

### MRI Relaxation Properties of Nanoparticles for Contrast Agent Compositions

In the instant example, exemplary nanoparticles comprising Fe₁₆N₂ can be characterized. Nanoparticles of various diameters and coatings (e.g., 3 nm, 5 nm, 8 nm, 12 nm, and 18 nm) as well as various substituents on the surface of the nanoparticles (e.g., dextran, polyethylene glycol, and functionalized silica) were evaluated. Magnetic resonance relaxivities of nanoparticles can be measured using a clinical 3T MR scanner with a headcoil. Further, IR-FSE sequence can be used to measure T1 properties using the following parameters: TR=4000 ms, TE=14 ms, and TI=25-3500 ms. CPMG sequence can be used to measure T2 properties using the following parameters: TR=5000 ms and TE=16-200 ms.

### EXAMPLE 6

### Evaluation of MRI Image Quality Using Nanoparticles for Contrast Agent Compositions in Healthy Subjects

The efficiency of nanoparticles for contrast agent compositions can be evaluated in healthy subjects. In the instant example, exemplary nanoparticles comprising Fe₁₆N₂ can be evaluated in rats. The contrast agent compositions comprising Fe₁₆N₂ can be diluted to 100 mg/mL and can be administrated to anesthetized rats intravenously via the tail vein at various concentrations (e.g., at 0.5, 0.2, 0.1, and 0 mL/kg body weight). The animals can then be scanned using MRI and the resulting images can be used to determine whether the nanoparticles enhance T1 relaxation in the circulating system of the animals.

The blood vessel signal persistence can be recorded. Blood pool imaging is important for the detection of various disease, for example myocardial infarction, renal failure, atherosclerotic plaque, thrombosis, and angiogenesis of tumor cells. Dynamic, time-resolved magnetic resonance and 3D-FLASH images of rats can be acquired using a wrist coil on an MRI scanner before and after injection of the contrast agents. The control and the contrasted images can be compared. The dynamic, time-resolved MR angiography can be obtained using an interpolated temporal resolution. Commercial MRI contrast agents can also be used in rats. The images generated from the Fe₁₆N₂ can be compared and evaluated with and commercially available gadolinium contrast agents. In addition, Ferumoxytol, an FDA-approved iron oxide nanoparticle used for anemia treatment, can be used as another comparative contrast agent.

### EXAMPLE 7

### Evaluation of MRI Image Quality Using Nanoparticles for Contrast Agent Compositions in Diseased Subjects

Another application of MRI is for diagnosis of disease, for instance cancer. The efficiency of nanoparticles for contrast agent compositions can be evaluated in diseased subjects. In the instant example, exemplary nanoparticles comprising Fe₁₆N₂ can be evaluated in inducible rat cancer models. In comparison, other treatment groups can include i) commercially available gadolinium contrast agents and/or ii) Ferumoxytol, an FDA-approved iron oxide nanoparticle composition.

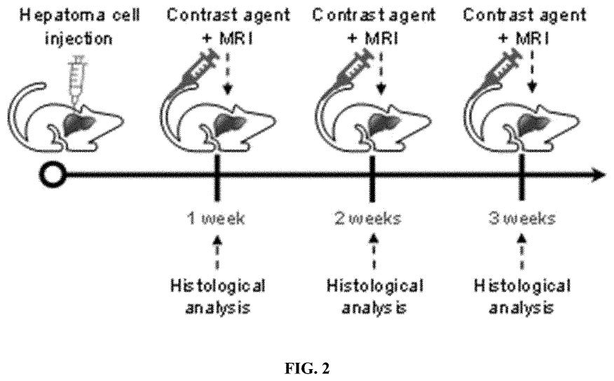

Liver cancer can be induced in rats for evaluation. A rat hepatocellular carcinoma cell allograft model can be prepared according to known methods (see, e.g., Guo, Y. et al., “Highly malignant intra-hepatic metastatic hepatocellular carcinoma in rats,” Am J Transl Res 2010;3:114-120 and Munoz, N. M. et al., “Comparison of dynamic contrast-enhanced magnetic resonance imaging and contrast-enhanced ultrasound for evaluation of the effects of sorafenib in a rat model of hepatocellular carcinoma,” Magn Reson Imaging 2019;57:156-164, both incorporated herein in their entirety). Briefly, Buffalo rats can be anesthetized and laparotomy performed to expose the left hepatic lobe. An injection of 1×10⁶ McA-RH7777 rat hepatoma cells suspended in PBS can be made under the hepatic capsule into the lobe. Fifty-four rats can receive McA-RH7777 cells, and 6 control rats can receive PBS only (60 rats total). The allograft-bearing rats can be randomized to receive one of three treatment groups. Each group will receive intravenous injection of one of the contrast agents described in the instant example above, followed by MRI with a 1.0T magnetic field 7, 14, and 21 days after tumor cell injection (see FIG. 2). Six rats from each contrast agent group plus 2 control rats can be scanned at each time point and humanely killed for histological analyses. The experimental design of the instant example is shown in FIG. 2.

Breast cancer can be induced in rats for evaluation. A breast cancer model in nude rats can be prepared according to known methods (see, e.g. Gupta, V. et al., “Repair and reconstruction of a resected tumor defect using a composite of tissue flapnanotherapeutic-silk fibroin and chitosan scaffold,” Ann Biomed Eng 2011;39:2374-2387 and Mishra, D. et al, “Silk fibroin nanoparticles and cancer therapy,” in: Mathur A. B., ed. Nanotechnology in Cancer. Amsterdam, Netherlands: Elsever Inc.; 2017, both incorporated herein in their entirety). Human GILM2 cancer cells can be injected into the mammary pads of 10 female nude rats. In some rats, the implanted GILM2 cells can grow into tumors larger than 50 mm³ within 8-10 weeks. The visible tumor from one of these rats can be collected, cut into smaller pieces (5 mm³), and implanted into the mammary pads of 54 female nude rats. Approximately 80% of these rats can develop breast tumors within 4 weeks.

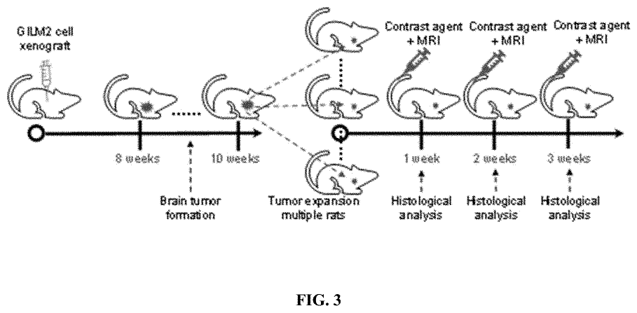

All tumor-bearing rats can be randomly divided into three groups. Each group will receive intravenous injection of one of the contrast agents described in the instant example above, followed by MRI with a 1.0T magnetic field strength. Tthe quality of the images obtained with the different contrast agents will be compared and the number of tumor-bearing rats identified by MRI with the contrast agents can be recorded. For confirmation of the MRI findings, breast tumor biopsies from each rat can be subjected to histological analyses. The experimental design is shown in FIG. 3.

Brain cancer can be induced in rats for evaluation. A brain cancer model in nude rats can be prepared according to known methods (see, e.g., Lal, S.; et al., “An implantable guide-screw system for brain tumor studies in small animals,” J Neurosurg 2000;92:326-333 and Lang, F. M. et al., “Mesenchymal stem cells as natural biofactories for exosomes carrying miR-124a in the treatment of gliomas,” Neuro Oncol 2018;20:380-390, both incorporated herein in their entirety). This xenograft model allows the growth of human glioma in the brains of rats. Compared with conventional chemical-induced brain tumor models, this xenograft model can be established within a significantly shorter amount of time (within 1 week) and with a high success rate (97%).

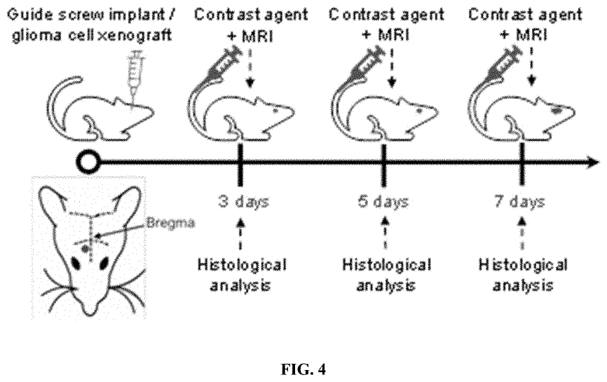

First, a 2.6-mm hollow guide screw can be implanted into a drilled hole about 1 mm anterior to the bregma (see FIG. 4). The guide screw has a 0.5-mm-diameter hole that allows a 26-gauge needle to be inserted into the caudate nucleus of the brain. Approximately 6×10⁵U87MG human glioma cells suspended in about 3 μl of PBS can be slowly injected into the caudate nucleus. The depth of the injection is controlled by the cuff attached to the needle.

After the injection, the needle can be removed, and the hole in the guide screw can be closed. The brain tumor should form within 4 weeks after injection. Fifty-four nu/nu rats will receive glioma cells, and 6 control rats can receive PBS only (60 rats total).

All xenograft-bearing rats can be evenly divided into three treatment groups. Each group will receive intravenous injection of one of the contrast agents described in the instant example above. On days 3, 5, and 7 after xenograft implantation, 6 rats from each treatment group and 2 rats from the control group can be injected with one of the 3 contrast agents, undergo MRI with a 1.0T magnetic field, and be humanely killed for histological analyses. The experimental design is shown in FIG. 4.

### EXAMPLE 8

### In Vitro Evaluation of Biosafety of Nanoparticles for Contrast Agent Compositions

The potential toxicity of nanoparticles for contrast agent compositions to human cell lines can be evaluated in vitro. For example, evaluation of the described contrast agent compositions can be made in the 293HT, HepG2, Caco-2, and A549 cell lines, which represent the kidney, liver, colon, and lung, respectively.

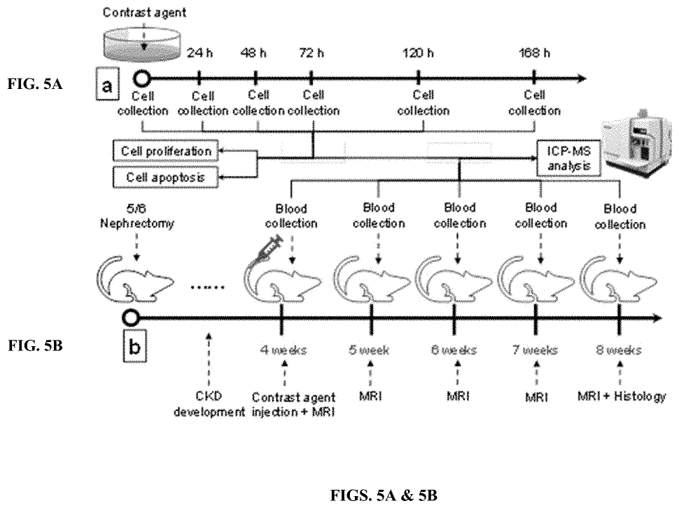

Cultures of each cell line can be incubated with nanoparticles comprising iron nitride, a gadolinium-based contrast agent, or Ferumoxytol at various concentrations (e.g., 0, 10, 50, 250, and 1000 ng/ml for 4 hours). Then the cells can be washed and continuously cultured in fresh media. Cell samples can be harvested from each group at 0, 24, 48, 72, 120, and 168 hours after treatment (see FIG. 5a).

Samples harvested at each time can be analyzed. First, cell proliferation and apoptosis can be assessed by immunofluorescence with Ki67 and TUNEL staining, respectively. Second, the potential inflammatory reaction caused by the contrast agent compounds can be assessed by quantitative PCR for selected pro-inflammatory cytokines, such as interleukin (IL)-1β, IL-6, IL-8, and CCL4. Third, the retention time of particles for each contrast agent in the cells can be assessed using inductively coupled plasma mass spectrometry (ICP-MS). Briefly, 5×10⁶ cells from each harvested sample can be washed with PBS and resuspended in deionized water. An equal volume of a buffer containing hydrochloric acid and nitric acid at a ratio of 3:1 can be added to the cell suspension. After evaporation, the contrast agent left behind can be quantified by ICP-MS. The experimental design for the instant example is shown in FIG. 5A.

### EXAMPLE 9

### In Vivo Evaluation of Biosafety of Nanoparticles for Contrast Agent Compositions

Since gadolinium contrast agents have demonstrated toxic effects to kidneys in humans, MRI procedures using gadolinium are not recommended for patients with severe kidney conditions. The efficiency of nanoparticles for contrast agent compositions can be evaluated in this patient population, for instance nanoparticles comprising Fe₁₆N₂ for rat acute kidney disease (AKD) models and for rat chronic kidney disease (CKD) models.

The rat CKD model can be created by nephrectomy, which surgically removes a large mass of kidney. The rat AKD model can be induced by warfarin following a nephrectomy based on the methods known in the art.

The Fe₁₆N₂ or Gadolinium agents will be administrated to the rats with CKD or AKD followed by MRI to evaluate the damage to the kidneys.

The effects of contrast agent compositions comprising Fe₁₆N₂ in comparison to contrast agent compositions comprising gadolinium will be examined in CKD rats and AKD rats by detecting the accumulation of the contrast agents in kidneys and other organs. In addition, evaluations including histological analyses on the different tissues and organs, such as kidney, liver, spleen, lung, skin, bone, and blood, can be made. The samples can be checked by a field emission scanning electron microscope with energy dispersive spectroscopy (SEM/EDS) followed by X-Ray microscopy. Pathological changes in the collected tissues caused by the contrast agents can be evaluated by histological microscopy. The experimental design for the CKD model of the instant example is shown in FIG. 5B.

What is claimed is:

1. A contrast agent composition comprising a plurality of nanoparticles, wherein one or more nanoparticles comprises an iron nanoparticle.

2. The contrast agent composition of claim 1, wherein the iron nanoparticle comprises iron nitride, iron oxide, or a combination thereof.

3. The contrast agent composition of claim 1, wherein the iron nanoparticle comprises iron oxide.

4. The contrast agent composition of claim 1, wherein the iron nanoparticle comprises iron nitride.

5. The contrast agent composition of claim 4, wherein the iron nitride comprises at least one phase of iron nitride selected from the group consisting of α-FeN (α expanded body-centered cubic (bcc)-Fe), α″-Fe₁₆N₂ (alpha double prime iron 16, also referred to as Fe₈N), γ′-Fe₄N, ϵ-Fe₃₋ₓN (0≤x≤1), ζ-Fe₂N, γ″-FeN, γ′″-FeN, and any combination thereof.

6. The contrast agent composition of claim 4, wherein the iron nitride comprises Fe₁₆N₂.

7. The contrast agent composition of claim 4, wherein the iron nitride comprises Fe₄N.

8. The contrast agent composition of claim 1, wherein the iron nanoparticle comprises i) a core comprising Fe₁₆N₂ and ii) a shell on the surface of the nanoparticle.

9. The contrast agent composition of claim 8, wherein the shell comprises Fe, FeO, or a combination thereof.

10. The contrast agent composition of claim 8, wherein the shell comprises Fe.

11. The contrast agent composition of claim 8, wherein the shell comprises FeO.

12. The contrast agent composition of claim 1, wherein one or more nanoparticles comprises one or more substituents on the surface of the nanoparticle.

13. The contrast agent composition of claim 12, wherein the substituent comprises an organic substituent.

14. The contrast agent composition of claim 12, wherein the substituent comprises an inorganic substituent.

15. The contrast agent composition of claim 12, wherein the substituent comprises a silicon-based substituent.

16. The contrast agent composition of claim 15, wherein the silicon-based substituent comprises SiO₂.

17. The contrast agent composition of claim 15, wherein the silicon-based substituent comprises a Si-based polymer.

18. The contrast agent composition of claim 12, wherein the substituent comprises a drug, an antibody, or a molecule for targeting a biomarker.

19. The contrast agent composition of claim 1, wherein the contrast agent composition further comprises a liquid.

20. The contrast agent composition of claim 19, wherein the liquid is a medium acceptable for use in magnetic resonance imaging (MRI).
